(19) United States
(12) Patent Application Publication
(10) Pub. No.: US 2022/0315873 A1
(43) Pub. Date: Oct. 6, 2022

(54) MULTI-CONNECTOR PORT

(71) Applicant: EPPENDORE SE, Hamburg (DE)

(72) Inventors: Sebastian Selzer, Aachen (DE); Karl Rix, Velbert (DE); Wolfgang Streule, Titz-Rödingen (DE)

(73) Assignee: EPPENDORE SE, Hamburg (DE)

(21) Appl. No.: 17/626,174

(22) PCT Filed: Jun. 19, 2020
(86) PCT No.: PCT/EP2020/067208

§ 371 Date: Jan. 11, 2022

(30) Foreign Application Priority Data
Jul. 12, 2019 (EP) 19186043.6

(51) Int. Cl. C12M 1/00; C12M 1/12; B67D 7/02

(52) U.S. Cl. CPC C12M 21/00; C12M 37/02; B67D 7/0288

(57) ABSTRACT

A multi-connector port provides an air connection for sterile air, at least one access arranged on a connecting piece, and at least one line arranged on the connecting piece. The access comprises a self-closing diaphragm.

### CROSS-REFERENCE TO FOREIGN PRIORITY APPLICATION

The present application claims the benefit under 35 U.S.C. §§ 119(b), 119(e), 120, and/or 365(c) of PCT/EP2020/067208 filed Jun. 19, 2020, which claims priority to European Application No. 19186043.6 filed Jul. 12, 2019.

### FIELD OF THE INVENTION

The invention relates to a multi-connector port and to a method for filling or withdrawal via a multi-connector port.

### BACKGROUND OF THE INVENTION

In laboratories, in particular, in laboratories for biological processes, there is an increasing need for automation solutions that increase reproducibility, can work with small volumes, and at the same time can be used flexibly. There are currently two groups of automated solutions for bioreactor systems: autosamplers and automated pipetting systems for small volumes or stand-alone individual solutions. Owing to the lack of standardized connections, autosamplers can only be used in special aseptic systems that are closed off with respect to the outside, they are furthermore limited to sample collection, and dead volumes normally remain in the sampler. In the field of biological processes, retention times in the sampler can also influence the sample. Automated pipetting systems for small volumes or stand-alone individual solutions have the disadvantage that they are generally only suitable for certain bioreactors or containers, and the work performed using these solutions must take place in workstations. In general, with these systems, it is not possible to work with single-use bioreactors either.

The invention is, therefore, based on the object of providing an improved solution that addresses the stated problems.

### SUMMARY OF THE INVENTION

According to a first aspect of the invention, said object is achieved by means of a multi-connector port comprising an air connection for sterile air and at least one connecting piece, at least one access arranged on the connecting piece, and at least one line arranged on the connecting piece, wherein the access comprises a self-closing diaphragm.

The invention is based on the recognition that automated additions and withdrawals are possible, in particular, in bioreactors and vessels without the need to work in sterile workstations or closed-off systems, and for a wide variety of embodiments of bioreactors and vessels, if the addition or withdrawal point is decoupled from the reactor or vessel. For this purpose, between the bioreactor and the liquid-handling device, use is made of a multi-connector port which can be connected to the bioreactor in sterile and leaktight fashion via at least one line and which provides accesses for the liquid-handling device, which are penetrable by the tips of the device whilst providing closure to the outside. Aside from pointed hollow needles, tips of the device are also understood to mean relatively blunt tips, such as pipette tips or male connecting pieces. In the context of this invention, penetration means not only piercing but also the pushing-open of self-closing diaphragms, for example, in swabable valves, also referred to as wipeable valves or needle-free diaphragm valves, by blunt tips, for example, male connecting pieces.

The invention thus enables automated, sterile work in non-sterile environments for the filling with or the withdrawal of materials, for example, in bioreactors. It is thus possible for both existing handling systems in a non-sterile environment and existing bioreactors or vessels to be used together with the multi-connector port for contamination-free withdrawal or addition.

Furthermore, the invention encompasses the recognition that dead volumes or contamination from previous addition or withdrawal processes can be prevented or minimized if the multi-connector port can be purged via a connection for sterile air, such that liquid remaining in the connecting piece of the multi-connector port or in the line can be conveyed into the reactor or the vessel. It is thus also possible to work with very small sample or addition volumes, because these can be fully utilized. The multi-connector port according to the invention and its use thus provide a solution that can be used flexibly, does not require a sterile environment, and avoids dead volumes.

The invention also encompasses the recognition that, with the multi-connector port according to the invention, a large number of working steps are made possible which, in the case of existing systems, could not be integrated into one solution. With the multi-connector port, it is thus possible, for example, to perform sample collection from a bioreactor, sample dispensing into microreaction vessels for subsequent analyses, sample dispensing into a collecting container, sample dispensing into an automated system, such as an analysis device, bleeding of a bioreactor (removal of cells from the bioreactor in order to control the cell concentration), and addition of a medium or of a media cocktail, for example, into a bioreactor. Furthermore, it is now possible to supply media or media cocktails for the bioprocess in an automated and time-controlled manner.

In one embodiment, the air connection comprises a further self-closing diaphragm and a sterile filter, in particular, a sterile filter with a pore diameter of less than or equal to 0.22 μm. With an air connection configured in this way, sterile air can be added in a simple manner via a tip, without the need for structurally coordinated connections to be provided on the multi-connector port and the sterile air supply. Alternatively, the air connection may also be configured with common air connection systems.

The air connection may either be designed such that sterile air can be drawn through it into a tip, for example, of a handling system, and is then released from the tip into the at least one access, the air connection is then separate and not fluidically connected to the at least one access, or the air connection is directly fluidically connected to the at least one connecting piece and thus to the at least one access.

In one embodiment, a side of the sterile filter facing away from the self-closing diaphragm is thus in contact with the outside air. If, in this embodiment, a tip is inserted through the self-closing diaphragm and outside air is drawn in by means of said tip via the sterile filter, the sterile air that is subsequently present in a reservoir belonging to the tip is thus available for application into the at least one access, and it is thus possible in a relatively simple manner for sterile air to be provided for a purging operation without the need for sterile air to be kept available. Alternatively, it is also possible for an air connection of a common air connection system, in which sterile air is present, to be provided, and for said sterile air to be drawn into the reservoir via the tip.

In alternative embodiments, the air connection is fluidically connected to the connecting piece, such that the at least one access, the air connection, and the at least one line are fluidically connected to one another via the at least one connecting piece. In this way, the access, connecting piece and line can be purged using air that is released into the air connection. Here, too, the air connection may both be configured as a connection of an air connection system and comprise a further self-closing diaphragm and a sterile filter, in particular, a sterile filter with a pore diameter of less than or equal to 0.22 μm. In the latter case, air is, for example, applied through the diaphragm via a tip and applied, having been filtered by the sterile filter, into the connecting piece.

In one embodiment, the multi-connector port comprises multiple accesses arranged on the connecting piece. The provision of multiple accesses allows, for example, the simultaneous addition of different media but also the simultaneous withdrawal of multiple samples.

In a further embodiment, the multi-connector port has exactly one access and exactly one line on each connecting piece. In this embodiment, the media added or withdrawn via the accesses are conducted entirely separately from the respective access to the connected vessel, for example, the bioreactor. It is thus possible, for example, for occurrences of mixing before entry into the bioreactor to be avoided. It is particularly preferred if the multi-connector port has exactly two connecting pieces on which in each case one access and one line are arranged. It is furthermore preferred if one access for an addition of media and one access for a withdrawal of media are formed. In this embodiment, samples can be withdrawn without the risk of adulteration by previously added media.

In one embodiment, the multi-connector port has a multiplicity of accesses and connecting pieces. In this embodiment, too, only one air connection is required, and this can be used for purging all accesses. It is particularly advantageous here if the air connection is configured such that sterile air can be drawn through it into a tip. This embodiment is particularly advantageous because it can be used simultaneously with a large number of bioreactors.

In further embodiments, the respective access and the respective line and also the air connection are fluidically connected via the respective connecting piece. Purging is thus possible in both accesses via one air connection, but the media are conducted between vessel and access separately from one another, such that occurrences of mixing or contamination are avoided.

The multi-connector port advantageously has a fixing device. By means of this, it is, for example, possible for connecting pieces to be mechanically connected to one another for better handling as a self-contained component. In particular, it is, however, also advantageous to use a fixing device which is configured to connect the multi-connector port to a handling system, in particular, a handling robot. In this way, the multi-connector port can be exactly positioned and held in position and automated devices such as a handling robot can utilize the accesses in automated fashion.

The multi-connector port is preferably designed to be sterilizable, wherein the sterilization can preferably be performed by autoclaving, irradiation, or with ethylene oxide. Sterile access via the multi-connector port can thus be realized by way of simple sterilization methods without the need to work in closed systems or workstations.

In one embodiment of the multi-connector port, the at least one line has a gas- and liquid-tight connection at its end facing away from the connecting piece. Via this connection, the multi-connector port can be easily and securely connected to the head plate of a bioreactor, for example. Standardized connections that are compatible with the greatest possible number of containers or head plates are particularly advantageous here. For example, it may involve a Luer lock connection or screw connections. In a very simple embodiment, the at least one line is configured as a simple hose which can for example be plugged into a connection of a head plate of a bioreactor.

It is additionally advantageous if the multi-connector port has a cap to cover the at least one access and/or the air connection. In this way, the access and/or air connection can be protected against contamination while they are not in use.

In one embodiment, the self-closing diaphragm is configured as a pierceable septum, that is to say, as a septum which, even when penetrated, is impermeable outside of the penetration and forms a sealed closure with the penetrating object. It is particularly preferred if the pierceable septum is impermeable to gases and liquids up to a pressure of 0.5 bar, even if the septum is penetrated by a hollow needle or a pipette tip, in particular by a hollow needle or pipette tip with a diameter of less than 1.5 mm, and even after multiple penetrations by such a hollow needle or pipette tip. In one embodiment, the pierceable septum has a slot, in particular, a cross-shaped slot, for penetration. The septum may, for example, be manufactured from silicone; septa of this type are already known from the prior art.

In an alternative embodiment, the access may, however, also be configured as a needle-free diaphragm valve comprising the self-closing diaphragm; such valves are known for example from U.S. Pat. Nos. 5,368,801 A, 7,947,032 B2, or WO 2013/158756.

According to a second aspect, the invention relates to a method for filling or withdrawal via a multi-connector port as claimed, comprising the steps: connecting the at least one line of the multi-connector port to a vessel, in particular. a bioreactor, cleaning an outer surface of the at least one access and/or an outer surface of the air connection, in particular. by wiping or rinsing with a cleaning agent comprising isopropanol, penetrating the self-closing diaphragm of the at least one access with a tip, in particular. in the form of a hollow needle, a pipette tip, or a male connecting piece, adding a medium into the vessel or drawing a medium out of the vessel via the tip, withdrawing the tip, applying sterile air.

The method according to the second aspect of the invention shares the advantages of the multi-connector port according to the first aspect of the invention.

The application of sterile air preferably comprises the following steps: penetrating the further self-closing diaphragm of the air connection with a tip, drawing in ambient air through the sterile filter via the tip into a reservoir that is connected to the tip, withdrawing the tip from the further self-closing diaphragm, penetrating the self-closing diaphragm of the at least one access of the multi-connector port with the tip, applying air from the reservoir into the access.

This method, in particular, in conjunction with a handling system, makes it easy to use sterile air for purging and thus to avoid dead volumes without the need for sterile air to be kept available. This means that work can be carried out even more flexibly and more independently of other infrastructure.

Alternatively, the application of sterile air via the air connection is performed by virtue of sterile air being applied via the air connection into the at least one connecting piece that is fluidically connected to the air connection, wherein either sterile air is supplied from the outside via the air connection or air is applied via a further self-closing diaphragm and a sterile filter into the at least one connecting piece and the at least one line arranged thereon and the at least one access.

In a preferred embodiment, after the application of sterile air, a final cleaning of the outer surface of the at least one access and/or of the outer surface of the air connection is performed. Here, the cleaning is preferably performed with isopropanol or similar cleaning fluids and/or a disinfectant. The cleaning step makes it easier to work with the multi-connector port even outside of sterile environments and nevertheless ensure filling and withdrawal under sterile conditions. It is preferred here if the multi-connector port comprises at least one cap and this is removed before the cleaning operation and/or is mounted after the final cleaning operation.

It is particularly advantageous if a medium is added via a different access and a different line than those via which medium is drawn out.

In one embodiment of the method, in which the tip is part of a handling system and the connecting piece has a fixing device, the multi-connector port is exactly positioned in relation to the handling system by means of the fixing device before the cleaning operation. This facilitates automated filling and withdrawal by means of the handling system.

According to a third aspect, the invention relates to a system comprising a multi-connector port according to the first aspect of the invention and a handling system with at least one tip, in particular, in the form of a hollow needle, a pipette tip, or a male connecting piece. Such a system allows automated, contamination-free filling and withdrawal in bioreactors or other vessels even without a sterile environment. In addition, the system according to the third aspect of the invention also shares the advantages of the multi-connector port and of the method according to the further aspects of the invention.

Here, the handling system may, in particular, be a handling robot or an automated liquid-handling system.

In one embodiment, the system furthermore comprises an encapsulating device for encapsulating the tip with respect to ambient air. With the aid of such an encapsulating device, withdrawal or addition can be carried out even more reliably, because, in this way, the tip does not come into contact with the ambient air and contamination can thus be even more effectively avoided. The encapsulating device may, in particular, be designed as a nozzle device for causing sterile air to flow around the tip, or as a self-closing casing.

In the case of a nozzle device for causing sterile air to flow around the tip, at least one nozzle is connected to a connection for sterile air and is arranged in the vicinity of the tip and directed toward the tip such that the sterile air flows around said tip, and said tip has no contact with the ambient air, at least over the length in which said tip can extend, at a maximum, into the at least one access or the air connection. A sterile air curtain is thus established by means of the nozzle device. The sterile air may be caused to flow around permanently, or at least during a penetration of the tip until it has penetrated fully, and for as long as the tip is not penetrating.

The self-closing casing encloses the tip in an air-tight manner when said tip is free, that is to say, is not penetrating anything; when the tip is penetrating for example into a self-closing diaphragm, said casing is pushed on, and said casing closes again upon a withdrawal. Here, the self-closing casing, which may, for example, be manufactured from silicone has, for example, a cross-shaped slot that extends over a certain length of the tip. If the tip is now pushed into a self-closing diaphragm, the casing is also pushed on; this then, together with the self-closing diaphragm, closes off the tip with respect to the ambient air. As soon as the tip is withdrawn, the casing wraps itself back around the tip. The self-closing casing may be designed, in particular, in the form of a cap, for example, a silicone cap with a cross-shaped slot.

In a further alternative, the air connection and/or the at least one access comprises a cap that can be mounted onto and removed from the tip in automated fashion.

### BRIEF DESCRIPTION OF THE DRAWINGS

Further embodiments will be discussed below by way of example on the basis of the accompanying Figures, in which:

FIG. 1 shows an embodiment of a multi-connector port according to the first aspect of the invention;

FIG. 2 shows a further embodiment of a multi-connector port according to the first aspect of the invention;

FIG. 3a shows a further embodiment of a multi-connector port according to the first aspect of the invention;

FIG. 3b shows a further embodiment of a multi-connector port according to the first aspect of the invention;

FIG. 4 shows a further embodiment of a multi-connector port according to the first aspect of the invention;

FIG. 5 shows an embodiment of a method according to the second aspect of the invention;

FIG. 6 shows an embodiment of a system according to the third aspect of the invention;

FIG. 7 shows a detail view of parts of an embodiment of a system according to the third aspect of the invention; and

FIG. 8 shows a detail view of parts of an embodiment of a system according to the third aspect of the invention in two states.

### DETAILED DESCRIPTION OF THE EMBODIMENTS

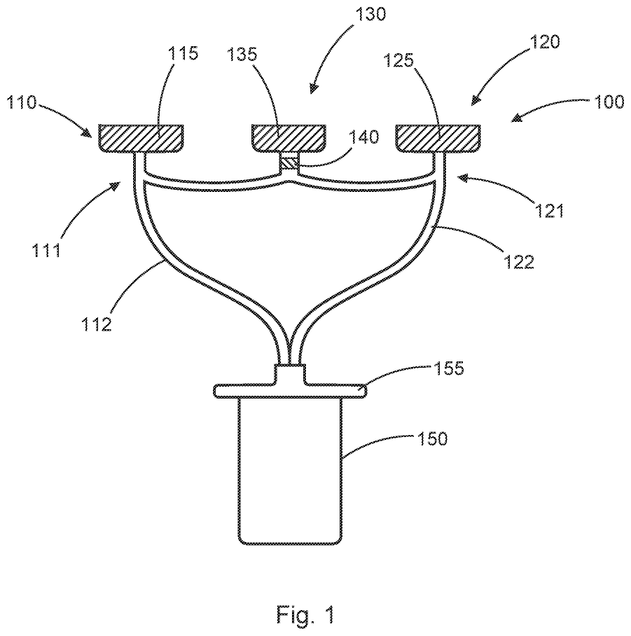

FIG. 1 shows an embodiment of a multi-connector port 100 according to the first aspect of the invention. In the embodiment shown, the multi-connector port 100 has two accesses 110, 120. Each of these accesses 110, 120 is arranged on a connecting piece 111, 121. Furthermore, in each case, one line 112, 122 is arranged on the respective connecting piece 111, 121. Each of the accesses 110, 120 has a self-closing diaphragm 115, 125. In the embodiment shown here, the self-closing diaphragm 115, 125 of each access 110, 120 is configured as a pierceable septum. The septum may, for example, be a commercially available silicone septum; this can be penetrated by a tip, for example, in the form of a hollow needle or a pipette tip, and yet seals off in leak-tight fashion with respect to the outside. In preferred embodiments, the pierceable septum is configured with a slot, in particular, a cross-shaped slot, through which the tip can penetrate, such that the walls of the slot lie tightly against the tip and thus close off the lumen under the septum in leak-tight fashion with respect to the outside. In alternative embodiments that are not shown here, the accesses 110, 120 may also be configured as needle-free diaphragm valves. Aside from the accesses 110, 120, the multi-connector port 100 has an air connection 130 which, in the embodiment shown, likewise comprises a further self-closing diaphragm 135 and a sterile filter 140. In the embodiment shown, the sterile filter has a pore diameter of less than or equal to 0.22 μm. In the embodiment shown, the air connection 130 is fluidically connected to the lines 112, 122 and the accesses 110, 120 via the connecting pieces 111, 121. With this embodiment of the air connection, purging of the connecting pieces 111, 121 and of the lines 112, 122 with sterile air is easy to realize by virtue of air being introduced via a tip through the further self-closing diaphragm and being filtered by the sterile filter 140. There is thus no need for sterile air to be kept available with corresponding connection pieces for the storage thereof, which can then be connected to the multi-connector port. Alternatively, it is also possible to provide standardized air connections for sterile air on the multi-connector port. The lines 112, 122 lead here to the head plate 155 of a bioreactor 150, for the filling and withdrawal of which the multi-connector port 100 is used here. The multi-connector port 100 may, however, also be used for a large number of other vessels and connection possibilities. It can be flexibly combined with other systems. For this purpose, special connections for connecting in leak-tight fashion to the respective vessel may be provided on the lines 112, 122. The line 112, 122 may, however, also be configured in the form of simple hoses which are inserted into, for example, existing septa on head plates.

The multi-connector port 100 allows simple and reliable filling and withdrawal for a large number of vessels by virtue of the addition and withdrawal points being decoupled from the vessel itself. The addition or removal takes place via the accesses 110, 120. In the embodiment shown here, it is particularly advantageous if one of the accesses, in this case the access 110, is only used for additions and the other access, in this case access 120, is only used for withdrawals. It can thus be ensured that samples taken from the vessel are not contaminated by media previously added via the access 110 and its connecting piece 111 and the line 112. The access 120 and its connecting piece 121 and the line 122 are fluidically separated from the access 110. The two parts of the multi-connector port are connected to one another only via the air connection, through which no liquids are exchanged. As already mentioned, the air connection 130 serves for the purging of the connecting pieces and lines such that any media present in the connecting pieces 111, 121 or lines 112, 122 are forced into the reactor by the addition of sterile air. Previously added media are thus transferred fully to the reactor and scarcely any dead volumes, or no dead volumes, remain within the connecting piece or the line. It can be ensured in this way that the previously set quantity of medium also actually reaches the bioreactor or the other vessel. In the case of the access for withdrawal, the medium withdrawn by way of the purging with sterile air is forced back into the bioreactor or the respective vessel such that no medium remains in the withdrawal line, which medium would over time be exposed there to conditions different than those in the reactor itself. It is thus ensured even for later withdrawals of samples that the samples originate entirely from the interior space of the bioreactor or of the other vessel, and are not contaminated by sample residues that have remained in the line or the connecting piece for a relatively long period of time. The entire multi-connector port 100 is preferably designed to be sterilizable. It is furthermore advantageous if all of the outer surfaces of the multi-connector port, in particular, the diaphragm, can be easily sterilized for example by wiping with a cleaning liquid such as isopropanol or else merely spraying or rinsing with such a cleaning liquid. By means of these cleaning and sterilization steps, it can be ensured that the filling of vessels is possible in a sterile manner even outside sterile working environments by means of the multi-connector port 100. This lowers the costs for the working steps and at the same time facilitates the work involved in the withdrawal or addition of samples, which can, therefore, also be performed flexibly in terms of location. As a further protective measure that is not shown here, the multi-connector port 100 may comprise one or more caps for the accesses 110, 120 and the air connection 130, which caps are firstly removed before an addition or withdrawal of media and are mounted again after the completion thereof, such that the accesses and the air connection are protected against contamination while they are not in use.

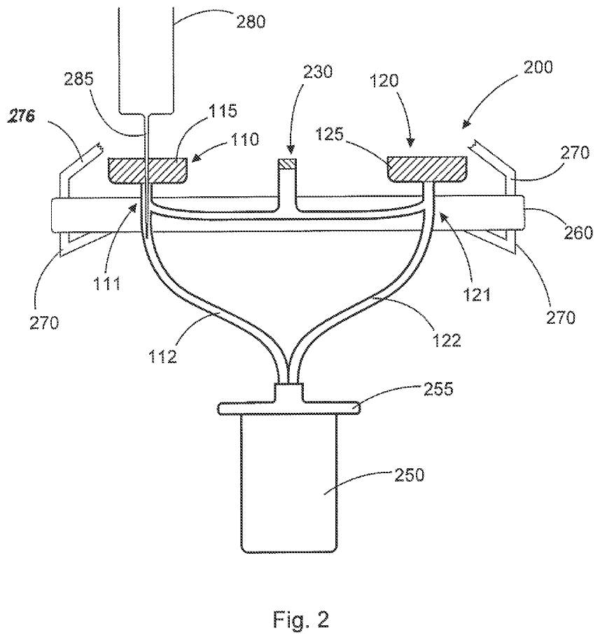

FIG. 2 shows a further embodiment of a multi-connector port 200 according to the first aspect of the invention. The multi-connector port 200 is of substantially identical construction to the multi-connector port 100 from FIG. 1. Therefore, the further features, in particular, will be discussed below, and reference is otherwise made to the description relating to FIG. 1. Components of the multi-connector port 200 that are identical to those of the multi-connector port 100 are denoted by the same reference designations. On the multi-connector port 200 is connected to the head plate 255 of a bioreactor 250. In the embodiment shown, however, said head plate is arranged in a liquid-handling system, by means of which automated additions and withdrawals can be realized. In the embodiment shown, the multi-connector port 200, therefore, has a fixing device 260 which, here, is connected and fixed to the handling system by means of brackets 270 belonging to the handling system, such that a handling robot can move easily and in an exact manner to the accesses 110, 120 of the multi-connector port 200. It is shown here by way of example how the tip 285 of a handling robot 280 penetrates the self-closing diaphragm 115 of the access 110. As can be seen, the tip 285, configured here as a hollow needle, penetrates through the diaphragm into the connecting piece 110, such that liquid can be released into the connecting piece 110 and its adjoining line 112 and thereby reaches the bioreactor 250. The multi-connector port 200 has an air connection 230 which is configured by way of a screw thread for the connection of a line for sterile air. By means of this connection, it is possible for a reservoir or a line for sterile air, which reservoir is already present at the respective workstation, to be used to purge the multi-connector port 200 and to force any residues out of accesses, connecting pieces, or lines into the connected bioreactor 250. The multi-connector port 200 otherwise shares the advantages that have been described with regard to the multi-connector port 100 in FIG. 1.

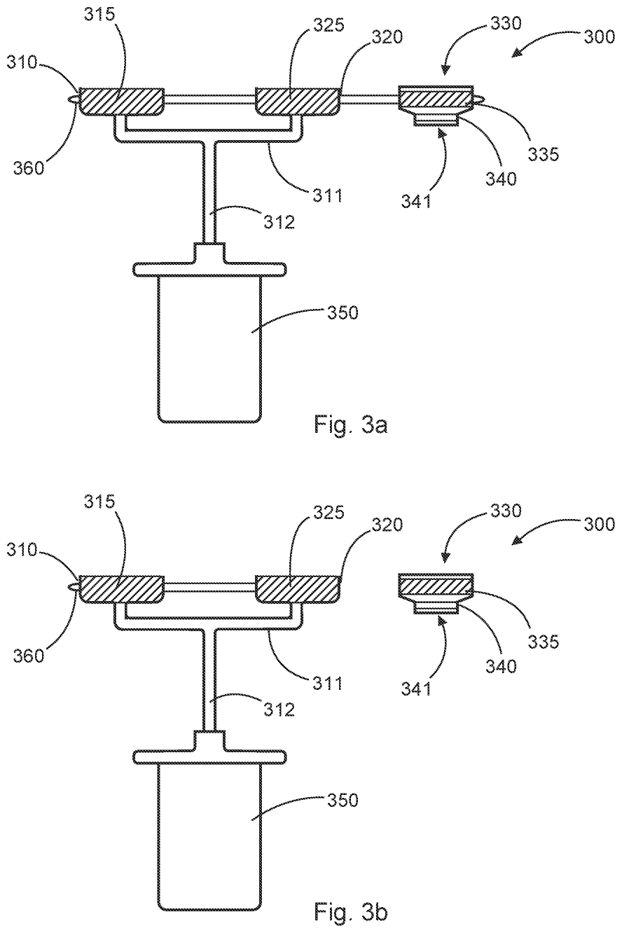

FIG. 3a shows a further embodiment of a multi-connector port 300 according to the first aspect of the invention. The embodiment shown here differs from that in FIGS. 1 and 2 in that, in the embodiment shown, only one connecting piece 311 is provided on which there are arranged two accesses 310, 320, each with a self-closing diaphragm 315, 325, and in that the air connection 330 with the further self-closing diaphragm 335 and the sterile filter 340 is in this case not fluidically connected to the connecting piece 311. In the embodiment shown, the sterile filter 340 is in contact with the outside air on its side 341 facing away from the further self-closing diaphragm. If a tip is now introduced from above through the self-closing diaphragm 330, then air can be drawn in through the sterile filter 340 via said tip and transferred into a reservoir connected to the tip. Following this, the tip can be introduced into one of the accesses 310, 320 and air can thus be applied into connecting piece 311 and line 312, such that medium situated in the access, connecting piece, or line from a previous filling or withdrawal process is forced into the reaction vessel 350 that is connected here. The multi-connector port 300 also has an air connection for sterile air 330, which air connection has a further self-closing diaphragm 335 and a sterile filter 340. Furthermore, the multi-connector port 300 has a fixing device 360 for integration into a handling system. The embodiment shown can be used, in particular, for vessels or intended purposes in the case of which there is little space available for the multi-connector port 300 and the use cannot result in problematic contamination in a common line or, for example, the intended purpose involves only additions or only withdrawals. To further reduce the space requirement, it is also possible for only one access, for example the access 310, to be provided, such that the multi-connector port 300 can be implemented in an extremely small space.

The embodiment shown in FIG. 3b differs from that in FIG. 3a merely in that the air connection 330 is not connected by means of the fixing device 360 to accesses 310, 320. The air connection 330 is configured separately here; this is particularly advantageous in embodiments (not shown here) of the multi-connector port which have a multiplicity of accesses, all of which can be purged by means of the one air connection 330. These embodiments of the multi-connector port allow a large number of bioreactors to be connected at the same time.

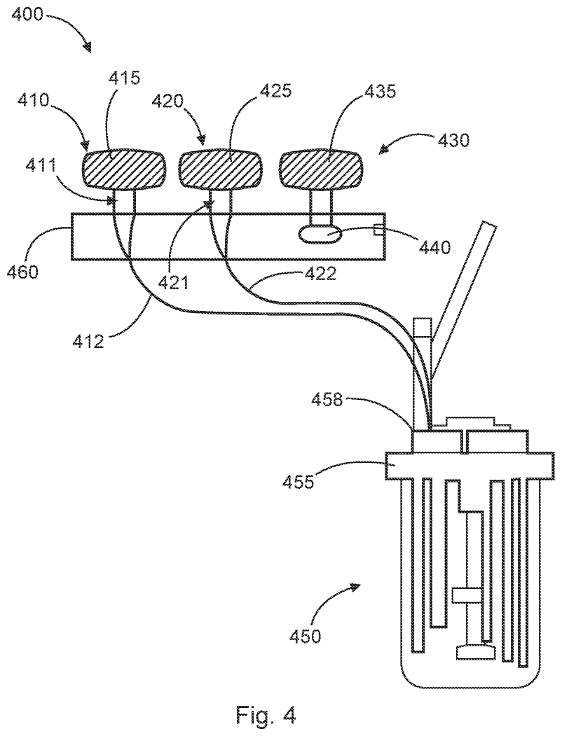

FIG. 4 shows a further embodiment of a multi-connector port 400 according to the first aspect of the invention. In the embodiment shown, the accesses 410, 420 are routed entirely separately from one another. A connecting piece 411, 421, on which in each case one line 412, 422 is also arranged, is arranged at each access 410, 420. Each of the accesses 410, 420 has a self-closing diaphragm 415, 425, in this case in the form of a pierceable silicone septum. The access 410 is preferably provided for addition and the access 420 for withdrawal, such that no adulteration occurs. The multi-connector port 400 furthermore has a fixing device 460 by means of which the multi-connector port 400 can easily be connected to a handling system. The fixing device 460 mechanically connects the accesses 410, 420 and the air connection 430 at the same time. As already described in detail with regard to the multi-connector port 300 and its air connection 330, said fixing device is equipped with a sterile filter 440 and a further self-closing diaphragm such that ambient air can be drawn in via the air connection 430 and sterilized in the filter 440 and can then, via a tip with a connected reservoir, be passed for purging into one of the previously used accesses. In the embodiment shown, the lines 412, 422 are plugged into a connection 456 in the head plate 455 of a bioreactor 450, which can be filled, and from which samples can be taken, via the multi-connector port 400.

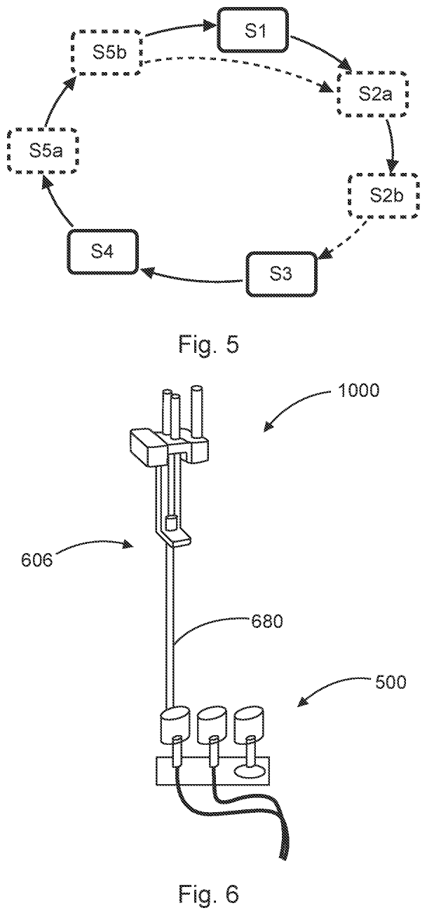

FIG. 5 shows an embodiment of a method according to the second aspect of the invention for filling or withdrawal via a multi-connector port. For preparatory purposes, in step S1, the at least one line of the multi-connector port is connected to a vessel, in particular, to a bioreactor. In the simplest case, the connection may be made by introducing the line, which is, for example, configured as a hose, into a diaphragm or a septum, for example, in a head plate of a bioreactor. If the multi-connector port is used in conjunction with a handling system, this step also includes exact positioning and optionally fixing in relation to the handling system, such that tips and other parts of the handling system can move easily and in an exact manner to the multi-connector port.

In the optional step S2a, a cap arranged over the at least one access or the air connection is removed; the cap is optionally cleaned beforehand, for example, with a cleaning agent and/or disinfectant. In step S2b, an outer surface of the at least one access and/or an outer surface of the air connection is then cleaned. This is preferably performed by rinsing, spraying, or wiping with a cleaning agent and/or disinfectant such as isopropanol. If the multi-connector port is integrated into a handling system, this step can be performed, in particular, in automated fashion by the handling system.

In step S3, the self-closing diaphragm of the at least one access is penetrated with a tip, in particular, in the form of a hollow needle, a pipette tip or a male connecting piece, and either a medium is introduced to the vessel, or a sample or a medium is withdrawn, via the tip.

In step S4, sterile air is applied into the previously used access of the multi-connector port. This may be performed via a fluidic connection between the air connection and the access or, in one embodiment, include the following sub-steps: penetrating the further self-closing diaphragm of the air connection with a tip; drawing ambient air through the sterile filter via the tip into a reservoir connected to the tip; removing the tip from the further self-closing diaphragm, penetrating the self-closing diaphragm of the at least one access of the multi-connector port with the tip, and finally applying air from the reservoir into the access.

In an optional step S5a, cleaning is then performed again, for example, by rinsing, spraying, or wiping with a cleaning liquid and, finally, if caps are used, these are mounted again in step S5b, and the caps are optionally sprayed with disinfectant.

If the multi-connector port is used further at the same location and with the same reaction vessel, this is then followed again by the optional step S2a. Otherwise, positioning of the multi-connector port, or connection to a further reaction vessel, is performed again in step S1.

FIG. 6 shows an embodiment of a system 1000 according to the third aspect of the invention. In the embodiment shown, the system 1000 comprises a multi-connector 500 comprising an air connection for sterile air and also at least one access arranged on a connecting piece and at least one line arranged on the connecting piece, wherein the access comprises a self-closing diaphragm as described in detail, for example, in FIGS. 1 to 4. The system 1000 furthermore comprises a handling system 606 with at least one tip 680, in this case in the form of a hollow needle. In the illustration shown, only part of the handling system is shown. With the system 1000, if the multi-connector port 500 is connected to, for example, a bioreactor, withdrawals or additions from or to this can now be performed in automated fashion in a non-sterile environment.

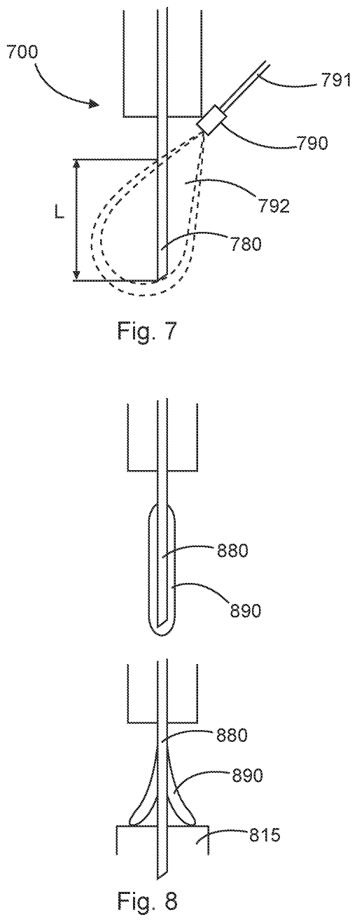

FIG. 7 shows a detail view of parts of an embodiment of a system according to the third aspect of the invention. Here, in detail, a tip 780 of a handling system 700 (not illustrated any further) is illustrated as part of the system, wherein the system comprises a nozzle device 790 with a nozzle for causing sterile air to flow around the tip 780. The nozzle is connected to a connection for sterile air 791 and is directed toward the tip such that sterile air is caused to flow around said tip, and said tip has no contact with the ambient air, over a length L. A sterile air curtain 792 around the tip is thus established by means of the nozzle device 790. The sterile air may be caused to flow around permanently, or at least during a penetration of the tip until it has penetrated fully, and for as long as the tip is not penetrating.

FIG. 8 shows a detail view of parts of an embodiment of a system according to the third aspect of the invention in two states. The system, parts of which are illustrated here, comprises an encapsulating device in the form of a self-closing casing 890, which is designed here as a silicone cap with a cross-shaped slot; here, at an end facing away from an open end of the tip 880, the silicone cap has an opening for receiving the tip, and at its opposite end facing toward the open end of the tip, said silicone cap has the cross-shaped slot. In the upper Figure, the tip 880 of the handling system of the system is free and encapsulated with respect to the ambient air by the self-closing casing. If, as illustrated in the lower Figure, the tip 880 is now pushed, for example, into a self-closing diaphragm 815, then the self-closing casing is pushed open when the self-closing casing 890 and the self-closing diaphragm 815 come into contact, such that a part of the tip 880 can penetrate into the self-closing diaphragm whilst the further parts of the tip 880 surrounded by the casing 890 remain protected by the latter. The casing 890 together with the self-closing diaphragm 815 then closes off the tip with respect to the ambient air. As soon as the tip 880 is withdrawn, the casing 890 wraps itself back around the tip 880.

1.-17. (canceled)

18. A multi-connector port comprising an air connection for sterile air, at least one access arranged on a connecting piece, and at least one line arranged on the connecting piece, wherein the access comprises a first self-closing diaphragm.

19. The multi-connector port as claimed in claim 18, wherein the air connection comprises a second self-closing diaphragm and a sterile filter.

20. The multi-connector port as claimed in claim 19, wherein the sterile filter has a pore diameter of less than or equal to 0.22 μm.

21. The multi-connector port as claimed in claim 19, wherein a side of the sterile filter facing away from the second self-closing diaphragm is in contact with the outside air.

22. The multi-connector port as claimed in claim 18, wherein the air connection is configured such that sterile air can be drawn through it into a tip.

23. The multi-connector port as claimed in claim 18, wherein the at least one access, the air connection and the at least one line are fluidically connected to one another via the at least one connecting piece.

24. The multi-connector port as claimed in claim 18, further comprising exactly two accesses, in which a first access is configured for an addition of media and a second access is configured for a withdrawal of media.

25. The multi-connector port as claimed in claim 18, further comprising a fixing device adapted for connection to a handling system.

26. The multi-connector port as claimed in claim 18, wherein the handling system comprises a handling robot.

27. The multi-connector port as claimed in claim 18, wherein the multi-connector port is adapted to be sterilizable, and wherein the sterilization is performed by autoclaving, irradiation, or with ethylene oxide.

28. The multi-connector port as claimed in claim 18, further comprising a cap for covering the at least one access or the air connection.

29. The multi-connector port as claimed in claim 18, wherein the first self-closing diaphragm is configured as a pierceable septum or wherein the access is configured as a needle-free diaphragm valve comprising the first self-closing diaphragm.

30. A method for filling or withdrawal via a multi-connector port as claimed in claim 18, the method comprising the steps:
connecting the at least one line of the multi-connector port to a vessel;
cleaning an outer surface of the at least one access or an outer surface of the air connection by wiping or rinsing with a cleaning agent comprising isopropanol;
penetrating the first self-closing diaphragm of the at least one access with a first tip in the form of a hollow needle, a pipette tip, or a male connecting piece;
adding a medium into the vessel or drawing a medium out of the vessel via the first tip;
withdrawing the first tip; and
applying sterile air.

31. The method as claimed in claim 30, wherein the vessel is a bioreactor.

32. The method as claimed in claim 30, wherein the application of sterile air comprises the following steps:
penetrating a second self-closing diaphragm of the air connection with a second tip;
drawing in ambient air through a sterile filter via the second tip into a reservoir that is connected to the second tip;
withdrawing the second tip from the second self-closing diaphragm;
penetrating the first self-closing diaphragm of the at least one access of the multi-connector port with the second tip; and
applying air from the reservoir into the access.

33. The method as claimed in claim 30, wherein after the application of sterile air, a final cleaning of the outer surface of the at least one access or of the outer surface of the air connection is performed.

34. The method as claimed in claim 33, wherein the multi-connector port comprises at least one cap and wherein the at least one cap is removed before the cleaning operation or is mounted after the final cleaning operation.

35. The method as claimed in claim 30, wherein the first tip is part of a handling system and the connecting piece has a fixing device and wherein the multi-connector port is exactly positioned in relation to the handling system by means of the fixing device before the cleaning operation.

36. A system comprising a multi-connector port as claimed in claim 18 and a handling system with at least one tip in the form of a hollow needle, a pipette tip, or a male connecting piece.

37. The system as claimed in claim 36, further comprising an encapsulating device for encapsulating the tip with respect to ambient air or a self-closing casing.

38. The system as claimed in claim 37, wherein the encapsulating device comprises a nozzle device for causing sterile air to flow around the tip.
